(19) United States
(12) Patent Application Publication
(10) Pub. No.: US 2024/0350015 A1
(43) Pub. Date: Oct. 24, 2024

(54) MULTI MODALITY ROTARY OPTICAL SYSTEMS AND METHODS OF THEIR USE

(71) Applicant: SpectraWAVE, Inc., Bedford, MA (US)

(72) Inventors: Eman Namati, Concord, MA (US); Tsung-Han Tsai, Newton, MA (US); Sean M. Pszenny, Arlington, MA (US); Kyle S. Darling, Acton, MA (US); Damon T. DePaoli, Cambridge, MA (US)

(21) Appl. No.: 18/681,694

(22) PCT Filed: Aug. 16, 2022
(86) PCT No.: PCT/US22/40409

§ 371 Date: Feb. 6, 2024

Related U.S. Application Data
(60) Provisional application No. 63/233,639, filed on Aug. 16, 2021.

(51) Int. Cl. A61B 5/00

(52) U.S. Cl. CPC A61B 5/0075; A61B 5/0002; A61B 5/0077; A61B 5/0084; A61B 2562/182

(57) ABSTRACT

Disclosed herein are characterization systems. A characterization system may comprise a stationary unit. The characterization system may comprise a rotary unit. optically connected to the stationary unit (e.g., via a FORJ) if present. The rotary unit may comprise a first optical channel, a second optical channel, and a light detector for detecting light for a first characterization modality. The light detector may be a camera, interferometer, or spectrometer. The first optical channel and/or the second optical channel may comprise a single mode optical fiber, a multimode optical fiber, or multiple waveguides. The first optical channel may be optically connected to the light detector. The stationary unit, if present, may be optically connected to the rotary unit at least in part with the second optical channel. The second optical channel may be used to detect light for a second characterization modality, with a detector in the stationary unit.

### PRIORITY APPLICATION

This application claims the benefit of U.S. Provisional Patent Application No. 63/233,639, filed on Aug. 16, 2021, the disclosure of which is hereby incorporated by reference herein in its entirety.

### TECHNICAL FIELD

This disclosure relates generally to methods and apparatus of detecting electromagnetic radiation for multimodal characterization of bodily lumens.

### BACKGROUND

In many applications, multiple sample characterization modalities are used to characterize a sample. Multimodal characterization can be performed using different forms of optical radiation from a single fiber or from a plurality of fibers. In the case of rotational characterization systems, it can be difficult to transport optical beams from more than one optical fiber over an optical rotary junction at high speed. Accordingly, reduced image quality can result from using optical rotary junctions in multimodal systems.

### SUMMARY

Sample characterization (e.g., of materials) can be performed in a rotary fashion using electromagnetic radiation (e.g., optical radiation) provided through a rotated probe. In rotary characterization systems, electromagnetic radiation often is provided using a stationary source of electromagnetic radiation (e.g., a light source) and is transmitted to a rotary probe via an electromagnetic rotary junction (e.g., a fiber optic rotary joint (FORJ)). Transmitting signals for more than one modality of optical characterization (e.g., interferometric imaging and diffuse spectroscopy) over a single FORJ can be complex and some solutions (e.g., multi-channel FORJs) can reduce performance of the characterization results (e.g., images, metrics, or diagnostics), especially at high rotational speeds.

Indeed, there are biomedical applications where high fidelity, rapid data and image gathering is important, such as intravascular characterization (e.g., imaging), where it may be desirable to characterize greater than 100 millimeters of a cardiovascular vessel in only a few seconds during a flush (e.g., contrast or saline) of the vessel. Current technology for rotational characterization only approaches these rates when a single characterization modality is used. Therefore, additional means to transmit signal for characterization modalities to and from stationary units and rotary units of a sample characterization system are critical for high-speed multimodal characterization. The present disclosure recognizes that using optical beams generated or detected, or both, on the rotational unit and transmitting detected information (e.g., by other forms of energy) to the stationary unit can allow for high-fidelity and high-speed multimodal characterization, thereby overcoming the challenges in prior optical systems.

Indeed, detecting electromagnetic radiation signals directly on rotary devices and transmitting their information by other means (e.g., wirelessly) is a solution to the short-comings of multi-channel FORJs. Separation of the modalities prior to fiber-optic junctions can reduce FORJ complexity, reduce device size, increase signal-to-noise ratio (SNR), improve manufacturability, and reduce system cost. These improvements can result in ameliorated characterization results (e.g., images, metrics, diagnostics) from the multiple signals (different signals may be electromagnetic radiation of different wavelengths, for example). The present disclosure provides, inter alia, a multimodal characterization system that can be used to rotationally transmit and detect electromagnetic radiation that has interacted with a tissue (e.g., a coronary artery) with high characterization sensitivity (e.g., an OCT system with greater than 90 dB, greater than 95 dB, greater than 100 dB, or greater than 110 dB sensitivity), at high rotational speeds (e.g., greater than 3,500 rpm, greater than 5,000 rpm, greater than 6,000 rpm, or greater than 10,000 rpm).

Rotary optical systems may include a stationary unit that houses stationary portions of a device (e.g., an interferometer), and a rotational unit that houses rotational portions of a device (e.g., a probe). A stationary unit may include a means to provide rotational torque to a rotary unit which may comprise a rotatable probe (e.g., the inner portion of an imaging catheter) for the purpose of transmitting light circumferentially to a portion of a sample (e.g., a lumen of an artery) in order to image or characterize internal bodily structures. Typically, light is transmitted from the light source to the rotary unit via a FORJ. A rotational probe typically comprises at least one optical channel (e.g., at least two optical channels) to carry light from the FORJ to the tissue and return through the FORJ to be detected by the stationary portion. A FORJ may have a maximum rotational speed up to which it is rated for operation. Current state of the art single channel FORJs that are commercially available can be rated for high-fidelity operation up to 20,000 rpm, have measured insertion losses less than 0.5 dB and insertion loss variation over rotation less than 0.5 dB. In the desired case of multimodal characterization wherein multiple signals are being transmitted to and from a sample, greater than one optical channel (e.g., non-coaxial waveguides) may be desired. FORJ's with greater than one optical channel are presently rated for much lower rotational speeds than single channel counterparts, limiting the speed of high fidelity multimodal characterization. For example, current state of the art FORJs for multi-channel operation are rated up to 3000 rpm for high-fidelity operation and include higher insertion loss and insertion loss variation (e.g., over a rotation). There also exist manufacturing advantages for using single channel FORJs such as robustness (e.g., longevity), reduced vibrations, reduced size and reduced cost. Also, multi-channel FORJs may be limited in the number of unique optical channels that can be multiplexed.

For certain characterization scenarios high fidelity, rapid characterization is required, such as is the case for intraluminal characterization during blood flushing. During such intraluminal characterization, probes rotate and translate rapidly (sometimes in excess of 10,000 rpm) to perform a characterization pullback. Speed of pullback is essential because optical characterization is often possible only while blood has been removed from the artery by the flush, for example, via radiopaque contrast material or saline. The pullback time scale is on the order of seconds (e.g., ˜2 seconds). Currently, multi-channel FORJs do not support high-fidelity characterization at these speeds. The present disclosure aims to provide a means to overcome these limitations to enable high-speed and high-fidelity multimodal sample characterization.

Given these challenges, one of the objectives of the present disclosure is to provide exemplary embodiments of systems, apparatus, and methods to perform rapid multimodal characterization. For example, multimodal characterization may include diffuse spectroscopy in conjunction with optical coherence tomography (OCT)/optical frequency domain imaging (OFDI—referred to herein interchangeably with OCT). Diffuse spectroscopy may include any form of spectral measurements where diffuse light (e.g., multiply scattered light) is measured. Diffuse spectroscopy may include, but is not limited to, reflectance intensity measurements, fluorescence spectroscopy, Raman spectroscopy, UV spectroscopy, visible spectroscopy, near infrared spectroscopy (NIRS), short-wave infrared spectroscopy (SWIRS) and infrared spectroscopy.

In order to perform multimodal analysis of luminal tissue, exemplary methods for performing OCT in combination with spectroscopy (e.g., NIRS) can be provided in a catheter. The exemplary method can employ exemplary apparatuses/devices/arrangements according to exemplary embodiments of the present disclosure to illuminate the tissue and collect the scattered light from the tissue.

In some embodiments, multimodal characterization is performed by direct detection of a characterization modality within the rotary unit of the device, before the FORJ, allowing high fidelity use of the single optical channel FORJ to be employed for another characterization modality. In some embodiments, the detection of a portion of the optical signal returning after interacting with a sample is performed within the rotary unit and the optical signal is converted to other forms of energy (e.g., electrical)). The detected signal may be transmitted to the stationary unit in the converted energy form or may be further converted prior to transmission (e.g., wireless radio frequency (RF)). Coupling a rotary detector with a collection optical channel may include a lens in the optical path, or may not (e.g., waveguide to detector direct coupling). In some embodiments, the detected signal within the rotary unit is continuously transmitted to the stationary unit during a characterization session. In some embodiments, the information carried by the detected signal may be temporarily stored on a computing storage device, within the rotary unit, until a characterization session has been completed and is then transmitted to the stationary unit (e.g., via an electrical docking contact.) In some embodiments, the detected signal is transmitted continuously during the characterization session using electrical rotary junctions (e.g., electrical slip ring.) In some embodiments, the detected signal may be wirelessly transmitted using a wireless transmission protocol (e.g., RF based) during characterization, or at the end of the characterization session. In some embodiments, electrical energy may be transmitted from the stationary unit to the rotary unit in order to generate light (e.g., LED) as a source emitter from within the rotary unit. In some embodiments, any combination of multiple rotary optical illuminators and rotary optical detectors may be deployed in order to perform any combination of multimodal characterization scenarios.

According to one exemplary embodiment, a first characterization modality subsystem may be a spectroscopy subsystem, for example a near-infrared spectroscopy (NIRS) subsystem. A second characterization modality subsystem may be an imaging subsystem, for example an optical coherence tomography (OCT) subsystem. Each system may use their own light source or may use the same light source. A system may be disposed such that light transmitted to the sample travels through a first illumination optical channel (e.g., a singlemode fiber) and the light received after interaction with a sample (e.g., a coronary artery) travels through more than one optical channel (e.g., a singlemode fiber and a multimode fiber). In some embodiments, light for both modalities may be transmitted through a single waveguide through a single-channel FORJ and detected via both the first waveguide (e.g., terminating in the stationary unit) and the second waveguide (e.g., terminating on the rotary unit) to provide separate optical characterization signals (e.g., interferometry and reflectance intensity.) In some embodiments, a sample characterization system is a catheter system, for example a cardiac catheter that can be used to rapidly characterize lumens (e.g., arteries) of a patient, in a multimodal manner.

In some embodiments, a characterization system comprises a rotary unit and, optionally, a stationary unit. If present, the stationary unit may be optionally optically connected to the rotary unit (e.g., via a FORJ). The rotary unit comprises a first optical channel, a second optical channel, and a light detector for detecting light for a first characterization modality. The light detector may be a camera, interferometer, or spectrometer. The stationary unit may be optically connected to the rotary unit at least in part with the second optical channel. The first optical channel and/or second optical channel may each, independently, comprise a singlemode fiber, a multimode fiber, or multiple waveguides (e.g., a multiclad fiber). In some embodiments, the first optical channel is optically connected to the light detector. In some embodiments, the second optical channel is used to detect light for a second characterization modality, e.g., different from the first characterization modality.

In some embodiments, a method for rotational sample characterization is used. The method may comprise providing illumination light to a sample through a second optical channel. The second optical channel, a first optical channel, and a first light detector may be rotated (e.g., by a motor). Signal may be collected from the sample through the first optical channel with a first light detector during the rotating. In some embodiments, second signal may be collected from the sample through the second optical channel with a second (e.g., stationary) light detector during the rotating. In some embodiments, the sample may be characterized with a first modality (e.g., NIRS) using the signal collected by the first light detector and the sample may also be characterized with a second modality (e.g., OCT) using the second signal collected by the second light detector.

In some embodiments, a characterization system includes a rotary unit and, optionally, a stationary unit. If present, the stationary unit may be optionally optically connected to the rotary unit (e.g., via a FORJ). The rotary unit may comprise a first optical channel, a second optical channel, and a light source for providing illumination light. The first optical channel may be optically connected to the light source. The stationary unit may be optically connected to the rotary unit at least in part with the second optical channel. The first optical channel and/or second optical channel may each, independently, comprise a singlemode fiber, a multimode fiber, or multiple waveguides (e.g., a multiclad fiber).

In some embodiments, a rotary unit for a sample characterization system comprises a rotatable housing. The rotary unit may further comprise a circuit board and a light detector or light source, or both, disposed on the circuit board. The circuit board may be attached to the housing, for example on an interior or exterior of the housing. The rotary unit may further comprise a first optical channel optically connected to the light detector or light source. The rotary unit may further comprise a second optical channel disposed through the housing along an axis. The circuit board may be disposed at least partially around the axis. If both a light source and a light detector are disposed on the circuit board, the first optical channel may optically connect to both the source and the detector (e.g., using different waveguides within the channel) or there may be an additional optical channel such that the source is connected to one channel and the detector is connected to a different channel.

In some embodiments, a multimodality optical device has a proximal face and a distal face, a proximal optical port on the proximal face, and a distal optical port on the distal face. The optical device may comprise a first optical waveguide configured to receive and transmit a first characterization modality. In some embodiments, the first optical waveguide makes an optical connection between the proximal optical port and the distal optical port. The optical device may further comprise a second optical waveguide configured to receive and transmit a second characterization modality. The second optical waveguide may be optically connected to the distal optical port. The second optical waveguide may be optically connected to a detector that may be housed within a rotary unit of the device.

Any two or more of the features described in this specification, including in this summary section, may be combined to form implementations not specifically explicitly described in this specification.

### BRIEF DESCRIPTION OF THE DRAWINGS

Drawings are presented herein for illustration purposes, not for limitation. The foregoing and other objects, aspects, features, and advantages of the disclosure will become more apparent and may be better understood by referring to the following description taken in conjunction with the accompanying drawings, in which:

FIG. 1 illustrates a conventional OCT imaging system with a stationary unit and rotary unit useful in understanding embodiments of the present disclosure;

FIG. 2 illustrates a multimodal OCT and spectroscopy imaging system with a stationary unit and a rotary unit housing a non-optical transmission device according to illustrative embodiments of the present disclosure;

FIG. 3 provides a detailed view of a conventional proximal rotary unit for an OCT system useful in understanding embodiments of the present disclosure;

FIG. 4 provides a detailed view of a proximal rotary unit with rotary detection and wireless transmission according to illustrative embodiments of the present disclosure;

FIG. 5 provides a detailed view of a proximal rotary unit with rotary detection and an electrical rotary junction according to illustrative embodiments of the present disclosure;

FIG. 6 provides a detailed view of a proximal rotary unit with rotary detection and docking station according to illustrative embodiments of the present disclosure;

FIG. 7 provides a detailed view of a proximal rotary unit with rotary illumination and detection as well as an electrical rotary junction according to illustrative embodiments of the present disclosure;

FIG. 8 provides a cross sectional view of a rotary circuit board with multiple detectors and a source according to illustrative embodiments of the present disclosure;

FIGS. 9A-9D provides a view of optical channel to rotary detector coupling methods according to illustrative embodiments of the present disclosure;

FIG. 10A provides a detailed view of a multi-channel catheter interconnect according to illustrative embodiments of the present disclosure.

FIG. 10B provides a detailed view of a multi-channel distal catheter tip according to illustrative embodiments of the present disclosure.

FIG. 11A provides an image of high-fidelity OCT and spectroscopy data acquired concurrently during a rapid rotation of a rotary optical system constructed in accordance with illustrative embodiments of the present disclosure.

FIG. 11B provides an image of high-fidelity OCT and spectroscopy data acquired concurrently during a rapid pullback of a rapidly rotating rotary optical system constructed in accordance with illustrative embodiments of the present disclosure.

### DEFINITIONS

In order for the present disclosure to be more readily understood, certain terms used herein are defined below. Additional definitions for the following terms and other terms may be set forth throughout the specification. In this application, unless otherwise clear from context or otherwise explicitly stated, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Light source”: as used herein, a “light source” refers to a source that provides (e.g., emits) light. Light is electromagnetic radiation (EMR) (e.g., photons). As used herein, light may have a frequency (wavelength) in a visible spectrum or not. A light source may emit one or more of visible light, near-infrared light, infrared light, long wavelength infrared light, ultraviolet light, deep ultraviolet light, and extreme ultraviolet light. In some embodiments, a light source may emit terahertz radiation. A light source may emit x-rays, microwaves, or radio waves. A light source may be, but is not necessarily, a laser. A light source may be, for example, a light source with reduced temporal coherence such as a source comprising a light emitting diode (LED) or a superluminescent diode (SLD). A light source may be a swept source, a tunable source or a narrowband source. In certain embodiments, a light source is a swept-source laser. In certain embodiments, a light source is a broadband source.

“Image”: as used herein, the term “image,” for example, as in a two-or three-dimensional image of tissue (or other sample), includes any visual representation, such as a photo, a video frame, streaming video, as well as any electronic, digital, or mathematical analogue of a photo, video frame, or streaming video. Any system or apparatus described herein, in certain embodiments, includes a display for displaying an image or any other result produced by a processor. Any method described herein, in certain embodiments, includes a step of displaying an image or any other result produced by the method. Any system or apparatus described herein, in certain embodiments, outputs an image to a remote receiving device [e.g., a cloud server, a remote monitor, or a hospital information system (e.g., a picture archiving and communication system (PACS))]. In some embodiments, an image is produced using a fluorescence imaging system, a spectroscopic imaging system, a luminescence imaging system, and/or a reflectance imaging system. In certain embodiments, a tomographic image and a spectroscopic image are co-registered to form a composite image. In some embodiments, an image is a two-dimensional (2D) image. In some embodiments, an image is a three-dimensional (3D) image. In some embodiments, an image is a reconstructed image. An image (e.g., a 3D image) may be a single image or a set of images. An imaging technique (e.g., using light provided by a light source) may produce one or more images.

“Probe”: as used herein, “probe” refers to a portion of a device or apparatus, or a subsystem, that directs light from one or more light sources toward a sample. A probe may comprise one or more optical elements, such as, for non-limiting examples, one or more lenses, one or more mirrors, and/or one or more waveguides (e.g., optical fibers). A probe may comprise any one or combination of one or more single mode fibers and one or more multi-mode fibers. For example, a probe may comprise one or more multi-clad fibers, such as a double clad fiber. A probe may comprise a housing (e.g., a sheath, for example, if the probe is part of a catheter).

“Sample”: As used herein, “sample” refers to matter to be characterized. Generally, any material, mixture, substance, or capable of characterization by a light can be used as a sample. A sample may comprise one or more materials. A sample may be gaseous, fluid, or solid. A sample may be, for example, a gel (e.g., a hydrogel), an elastomer, or a composite. A sample may be a biological sample. For example, a sample may be an organ or biological structure (e.g., tissue) or portion thereof. A sample may be an in vivo organ or in vivo tissue. For example, a sample may be an in vivo artery or portion thereof. A sample may comprise one or more features of interest. For example, a feature of interest may be, for example, arterial plaque (e.g., a vulnerable plaque, for example having a fibrous cap).

“Spectroscopy”: as used herein, “spectroscopy” refers to any form of characterization of a sample with a light source. A light source may have a narrowband (e.g., less than 2 nm) wavelength range (e.g., 1210.01-1210.02 nm, e.g. 1210 nm-1212 nm), a broadband wavelength range (e.g., 1160 nm-1280 nm), or more than one non-contiguous bands of wavelengths (e.g., 1205 nm-1215 nm and 1260 nm-1360 nm). For example, “visible spectroscopy” may refer to characterizing (e.g., imaging) a sample at a visible wavelength (e.g., 550 nm). As another example, “near infrared spectroscopy/NIRS” may refer to characterizing (e.g., imaging) a sample at a NIRS wavelength (e.g., 1210 nm). In some embodiments, scanning any source over any area of a sample can produce an image, and, this process may still be termed spectroscopy as the image pertains to the absorption and scattering characteristics of the sample in a specific wavelength range.

“Optical” is not limited to referring to visible light. For example, an optical channel may be constructed to transmit light having a frequency (wavelength) outside of the visible spectrum, such as infrared or ultraviolet light. Similarly, “optical detection,” “optical modality,” and other similar terms can utilize light (electromagnetic radiation) outside of the visible spectrum, such as infrared or ultraviolet light. Two components that are “optically connected” may be directly optically connected or have one or more additional optical components (e.g., waveguide(s), lens(es), beam splitter(s), multiplexer(s)) and/or free space disposed therebetween along an optical path.

### DETAILED DESCRIPTION OF CERTAIN EMBODIMENTS

Intraluminal characterization generally comprises rapidly rotating a light source to image the inner circumference of an internal object, as is performed in typical catheter-based intravascular characterization systems. Such a rotary optical system may comprise at least one light source meant for at least one mode of tissue characterization. In some cases, rotary optical systems may use a single optical channel (e.g., a singlemode fiber) to perform single-modality characterization or multi-modality sample characterization. In other cases, multi-channel optical systems (e.g., a singlemode optical and a multimode optical fiber) are required for sample characterization, for example, when characterizing structural properties (e.g., by imaging, for example with OCT) and absorption properties (e.g., with diffuse spectroscopy).

Tissue characterization may be performed with non-interferometric or with interferometric characterization, or both. In the case of interferometric characterization (e.g., OCT imaging) there may exist a beam splitter (e.g., a polarization beam splitter, half-mirror, cube splitter, or plate splitter) to split a light source into a sample and reference arm. The sample arm light may interact with a sample and the reference arm light may interact with a reference reflector. After reflection, the sample and reference light may be recombined, interfered, and directed to at least one detector (e.g., at least two) via a beam re-director (e.g., a circulator, e.g., fiber coupler, e.g., beam-splitter, e.g., a polarization beam splitter, etc.) to create an interference pattern that can then be detected. Detection may be performed using a spectrum-separating detection unit (e.g., a spectrometer) when deploying broadband light sources (e.g., spectral domain OCT (SDOCT)), or a time-domain detection unit (e.g., at least one photodetector) when deploying swept sources (e.g., swept source OCT (SSOCT)). Broadband light sources may also be deployed in conjunction with time-domain detectors (e.g., time-domain OCT (TDOCT), Light Detection and Ranging (LiDAR)). From a detector, analog signal may be converted to digital format and may be stored and processed on a computer storage device to display a characterization result (e.g., a spectral measurement, e.g., an image) or may be transmitted to the computer storage device to display the characterization result.

Non-interferometric characterization (e.g., NIRS) may use a narrowband light source (e.g., single wavelength), a broadband light source, or a tunable wavelength light source. The light source may be transmitted and detected through at least one (e.g., at least two) optical channels. Detection may be performed using a spectrum-separating detection unit (e.g., a spectrometer) or a time-domain detection unit (e.g., at least one photodetector, e.g., a CMOS, e.g., a charge coupled detector (CCD)) or any combination of more than one of these. Importantly, when light irradiates tissue, reflection is not the only optical phenomena that may occur. A detector may be disposed, at least in part, to detect light that has been reflected, refracted, scattered, transmitted (e.g., a non-absorbed portion of initially incident light), or produced [e.g., by photoluminescence (e.g., fluorescence, phosphorescence, or Raman emission), chemiluminescence, or bioluminescence)] by a sample in response to light from any light source impinging on the sample.

A combination of characterizations may be performed. In some embodiments, a single light source may be used to perform multi-modal characterization. In some embodiments, multiple light sources may be used to perform multi-modal characterization. In some embodiments, sources may be combined into a single optical channel using a light combining device (e.g., a polarization beam splitter, wavelength division multiplexer).

To transmit the light to the sample in a rotary fashion, a FORJ may be used to couple light from between a stationary waveguide and a rotary waveguide. A rotary waveguide may be rotated by a rotation device, such as a motor (e.g., direct drive, e.g., via a drive belt) and the ensemble (e.g., stationary waveguide, FORJ, motor and rotary waveguide) may be longitudinally displaced (e.g., using an actuator) to scan (e.g., image) a section of a sample. In the case of an intravascular probe (e.g., a catheter), another rotatable waveguide (e.g., a probe) may be connected in series to a rotary waveguide and may be housed in a non-rotary protective tubing (e.g., a sheath). A drive shaft may be used to transfer the torque from the proximal end of the probe to its distal tip.

The present disclosure also provides methods to improve robustness of rotary optical systems by reducing free space optics. In some embodiments, rotary units transmit optical energy to and from the rotary probe and stationary system using waveguides (e.g., reduced free space beam directors). For example, the FORJ may be connected to a multi-use optical fiber (e.g., permanently connected, for example mounted, glued, or cemented) which connects in series (e.g., optically coupled) to a waveguide within a single-use multi-channel rotary probe. In some embodiments, a 0 dB connector may be deployed at a fiber connection point to improve the serviceability of the system. Furthermore, a rotary detector may have another multi-use waveguide physically in contact with it (e.g., glued or cemented) which connects in series to a waveguide in a single-use multi-channel rotary probe. Such direct contact may improve signal collection efficiency.

In some embodiments of the present disclosure, at least one light source is provided in a stationary unit and generates light that is transmitted to a rotary unit via a single-channel FORJ. A rotary unit may house at least one optical channel, with at least one optical detector, as well as a means to transmit the detected signal to the stationary unit. In order to transmit light to the sample, an optical channel may be extended via a connection (e.g., a fiber interconnect) to a rotary probe. In some embodiments, light transmitted to the sample may travel through a first illumination optical channel and the light received after interaction with a sample may travel through at least one optical channel (e.g., the illumination optical channel and the collection optical channel.) In some embodiments, source light for both modalities may be transmitted from the stationary unit, through a single-channel FORJ, and detected via both the illumination optical channel (e.g., detecting in the stationary unit, for a first characterization modality) and a collection optical channel (e.g., detecting on the rotary unit, for a second characterization modality) to provide separate optical characterization signals (e.g., interferometry and reflectance intensity). Such an arrangement may be particularly beneficial where there is a large difference in intensity between signals being detected for different characterization modalities.

In some embodiments, the rotary detected signal may be transmitted to a stationary receiver via contacts (e.g., electrically conductive) that are connected while the rotary unit is stationary (e.g., while characterization (e.g., imaging) is not being performed). In some embodiments, the rotary detected signal may be stored in the rotary unit temporarily. In some embodiments, the detected signal may be transmitted wirelessly by a wireless means (e.g., RF, Bluetooth). In some embodiments, the detected signal may be re-transmitted wirelessly by emitting optical pulses/flashes, for example in free space (e.g., with no physical contact), that carry information about the detected signal, using a rotary optical emitter (e.g., an LED) and a stationary detector (e.g., a photodetector). In some embodiments, the detected signal may be transmitted to a stationary receiver via an electrical rotary junction (e.g., a slip ring) while the rotary unit is rotating (e.g., while characterization is being performed) or while the rotary unit is stationary. In some embodiments, an electrical rotary junction may provide power while the rotary unit is rotating but transmit detected signal while it is not rotating (e.g., after characterization is performed (e.g., after a catheter pullback)) for improved signal fidelity. In some embodiments, timing and/or acquisition signals may be transmitted from the stationary system back to the rotary unit, for example wirelessly through a wireless transmitter in the rotary unit. In some embodiments, the detected signal may be re-transmitted optically using an aligned rotary source and stationary detector (e.g., an optical slip ring). In some embodiments, the detected signal may be re-transmitted optically using an un-aligned rotary source and stationary detector, wherein portions (e.g., reflections) of the rotary optical source are enough to carry information on the detected signal. In some embodiments, the detected signal may be re-transmitted optically and detected using a stationary, circular (e.g. annular) active area photodiode. In some embodiments, the detected analog signal may be converted to a digital format prior to any form of rotary-to-stationary transmission. In some embodiments, further signal processing may occur within the rotary housing using a rotatable processing unit (e.g., field programmable gate array (FPGA)). In some embodiments, unprocessed signal, processed signal or both may be temporarily stored within the rotary unit using a miniature computer storage device (e.g., microcontroller) or other non-transitory memory (e.g., flash memory, read-only memory (ROM), erasable programmable ROM, or electrically erasable programmable ROM).

In some embodiments, components on the rotary unit may require energy (e.g., power) to operate. In some embodiments, the rotary unit may house an energy storage device (e.g., a battery) that is charged while the rotary unit is not in motion (e.g., via electrical contacts). In some embodiments, the rotary unit may house an energy storage device (e.g., a battery) that is charged while the rotary unit is in motion (e.g., via a wireless charging device). In some embodiments, energy may be provided to the rotary unit via an electrical rotary junction (e.g., a slip ring). In some embodiments energy may be provided to the rotary unit by inductive processes.

In some embodiments, more than one source of light may be detected with a single detector housed on the rotary unit. In some embodiments, there may exist more than one illumination optical channel and/or more than one collection optical channel. For example, in some embodiments there may be a single illuminating optical channel with more than one collection optical channels, each terminating at separate detectors housed within the rotary unit. In some embodiments, more than one collection optical channel may terminate on the same rotary detector after a combining means (e.g., a fiber coupler). In some embodiments, an illuminating optical channel may be split and directed to more than one location on the sample, for example at a distal end of a probe.

In some embodiments, multiple collection channels may be positioned to characterize separate locations, for example due to the relative position at a distal end of a probe. In some embodiments, any single collection optical channel may be split (e.g, using a wavelength-division-multiplexer (WDM)) and directed to more than one optical detector within the rotary unit (and/or outside the rotary unit). In some embodiments, portions of the collection optical channels may be combined (e.g., via a fiber coupler) with the illumination optical channel and optically transmitted back over a FORJ (e.g., a single-channel FORJ), for detection within the stationary unit, while other portions are detected on the rotary unit. In some embodiments, an optical fiber with multiple waveguides (e.g., coaxial waveguides) may serve as the illumination and collection optical channel within a rotary probe. In some embodiments, a multi-waveguide optical fiber may split portions of the signal from either of its waveguides, or both, and detect some portion of the optical signal on the rotary unit, allowing for single optical channel probes.

Optical noise, for example, originating from slight alignment variations during rotation of the FORJ, may affect the SNR of the detected signal. To overcome this, in some embodiments, optical common-mode rejection may be performed by splitting an illumination optical channel and performing simultaneous detection with the light returning from a collection optical channel. In some embodiments, portions of the light from an illumination optical channel may be split to provide a reference measurement for calibration, either before a FORJ (e.g., within in a stationary unit) or after (e.g., within a rotary unit.) Electrical noise also may exist, even after minimization of optical sources of noise. Therefore, in some embodiments, prior to transmission of a rotary detected signal, electrical waveforms may be adjusted, cleaned, and/or amplified by electrical means (e.g., using electrical circuits) for SNR enhancements. In some embodiments, devices used for signal improvement may include at least one transimpedance amplifier. In some embodiments, devices used for signal improvement may include gain circuits or level offset circuits, or both. In some embodiments, devices used for optical detection may include a means to optically isolate the optical components to minimize optical interference. In some embodiments, devices used for optical detection may include a means to electrically isolate the electrical components to minimize electrical interference. Reductions in electrical noise ultimately allow for higher fidelity measurements. In some embodiments, electrical isolation may include a faraday cage (e.g., an enclosed metal encasement, e.g., an aluminum block). Still, it can remain difficult to fully insulate electrical components over distances and unintended electrical interference may occur. Therefore, in some embodiments, devices used for signal quality maintenance (e.g., a single to differential circuit) may be used to preserve waveform fidelity during transmission of the detected signal. In some embodiments, a detected analog signal may be converted to a digital signal (e.g., via an analog to digital converter (ADC)) prior to transmission. Such processing units may increase complexity of the rotary circuit but could provide higher fidelity measurements, for example by simultaneous processing at the point of detection. In some embodiments, a signal is processed after transmission (e.g., to a remote computing device).

Collection optical channels may be coupled to a rotary detector in several ways that optimize light collection, manufacturing complexity and device cost. In some embodiments, active areas of a photodetector may be optically coupled directly (e.g., physically touching or stabilized in close proximity) to a collection optical channel (e.g., multimode optical fiber). Indeed, there may be signal collection improvements in direct coupling scenarios (e.g., owing to reduced losses on intermediate optical components) that can improve SNR. In some embodiments, collection optical channels may be aligned with beam shaping optics in order to optimize the collection of light from an optical channel onto the detector. Therefore, in some embodiments, a focusing, diverging, or collimating lens may be placed between a collection optical channel and a light detector. Likewise, in some embodiments wherein a spectral detector (e.g., spectrometer) may be used (e.g., spectrometers), spectrally diverging element(s) (e.g., fiber Bragg grating(s)) may be employed between a collection optical channel and the detector.

In some embodiments, at least one light source disposed within a rotary unit. In some embodiments, there may be multiple sources originating in a stationary unit. In some embodiments, there may be multiple sources on a rotary unit. Furthermore, it is understood that, in certain embodiments, a portion of returning light from any source may also be detected in a stationary unit via an illuminating optical channel.

In some embodiments, light sources may be modulated to lower and higher intensity states (e.g., on and off, e.g., bright and dark) to provide alternating modality characterizations, for example to allow for use of different wavelengths for different characterization modalities along a single optical channel. A light source may alternate between a bright phase and a dark phase (e.g., alternating between on and off) during operation at a rate of at least 10 Hz, at least 100 Hz, at least 1 kHz, at least 2 kHz, at least 5 kHz, at least 10 kHz, at least 15 kHz, at least 20 kHz, at least 50 kHz, at least 75 kHz, at least 200 kHz, at least 1 MHz or at least 10 MHz (e.g., and no more than 10 GHz, no more than 5 GHz, no more than 2 GHZ, no more than 1 GHz, no more than 500 MHz, no more than 250 MHz, no more than 100 MHz, no more than 50 MHz, no more than 10 MHz). In some embodiments, a light source may alternate between a bright phase and a dark phase at a frequency that corresponds (e.g., proportionately) to a rotational frequency of a probe, for example in a catheter, such as a cardiac catheter. If a light source is a swept source, the light source may be scanned across its wavelength range during a bright phase and, optionally, may be cycled during a dark phase.

In some embodiments, wavelength selective filters (e.g., high-pass, e.g., low-pass, e.g., dichroic, etc.) may be employed to provide multimodal characterization with simultaneous illuminations (e.g., at least two sources in a concurrent bright phase). In some embodiments, polarization-filtering means (e.g., a polarization beam splitter) may be employed to provide multimodal characterization. In some embodiments, a camera (e.g., one or more containing Bayer filters) (e.g., a CMOS camera) may be used as a rotary detector. In some embodiments, spectrometers may be used as a rotary detector to separate wavelength-dependent reflection signals.

When adding components to a rapidly rotating device, balance and air flow are important factors to minimize vibrations. In some embodiments, circuit boards are fabricated in such a way that optical components and electrical components disposed on a circuit board and/or in optical and/or electrical communication with the circuit board are weight balanced to minimize vibrations within a rotary unit. In some embodiments, vibration minimization may be performed by designing structural components of the rotary unit (e.g., a rotary device housing) to account for any imbalances (e.g., in combination with a balanced circuit board). In some embodiments, vibration minimization may be performed by adding other weight balancing components. In some embodiments, a circuit board may be circular (e.g., annular). In some embodiments the circuit board may be centered on the rotary axis. In some embodiments, there may be more than one circuit board within the rotary unit. For example, there may be two stacked circuit boards (e.g., circular circuit boards with hollow centers) located within the rotary unit (e.g., within a rotary cylindrical tube). In some embodiments, a circuit board may be situated on the exterior of the rotary unit (e.g., attached to the rotary housing).

It is advantageous to have modular systems, for example to separate devices that come into contact with a patient from devices that do not. As such, in some embodiments, a rotary unit may attach to detachable components (e.g., a probe, such as in a catheter) to allow for sterile workflows. In some embodiments at least a portion of a rotary unit (e.g., a rotary probe) may be housed within such a detachable component. In some embodiments, a detachable component and its contents are single use, such as a rotary probe (e.g., a cardiac catheter). In some embodiments, the detachable component and/or the rotary housing may comprise an information storage device (e.g., a microchip) that transmits information between them either wirelessly (e.g., via RF, such as in radio frequency identification (RFID)), and/or via a mechanical contact (e.g., a lever), and/or via an electrical contact. In some embodiments, this information informs which of the system's multi-modalities should be used in the given characterization session. In some embodiments, this information informs other aspects concerning the sources and detectors modulation during the characterization session (e.g., how many sources or detectors to use, e.g., at what time to use a given source or detector, e.g., what duty cycle of bright and dark phases should be used for each source or detector).

The present disclosure also includes methods to improve sensitivity of rotary optical system by reducing free space optics (e.g., reducing optical interfaces which may incur losses) and improve longevity by using sacrificial connectors (e.g., reducing damage to permanent fiber components). In some embodiments, rotary units transmit optical energy to and from the rotary probe and stationary system using mostly waveguides (e.g., reduced free space beam directors). In some embodiments each optical channel consists of more than one waveguide in series. For example, the FORJ may be connected to a multi-use optical fiber (e.g., permanently connected) which may then connect via an interconnect (e.g., via a single port of a duplex interconnect) to a single-use waveguide within a rotary probe. Furthermore, a rotary detector housed in a rotary unit may have another multi-use waveguide physically in contact with it, which connects via an interconnect (e.g., via a single port of a duplex interconnect), to a single-use waveguide in a rotary probe. In the case of a multi-channel probe a multi-channel interconnect (e.g., a duplex interconnect) may be used to optimize automized connections (e.g., it may be easier to align and connect a single multi-port interconnect, in an automated fashion, rather than multiple single single-port interconnects). In some embodiments, a multi-channel probe with more than one optical channel to connect, multiple single-port interconnects may be attached (e.g., glued or cemented) to each other to imitate the advantages a multi-port (e.g., duplex) interconnect. In some embodiments, a sacrificial low-loss connector may be used in-between segments of an optical channel (e.g., to improve device longevity). In some embodiments, multi-use rotary waveguides may optically connect the FORJ to an eccentric waveguide within the rotary probe. In some embodiments, a multi-use rotary waveguide may optically connect light from an eccentric waveguide within the rotary probe directly to a rotary optical detector. In some embodiments, a multi-use rotary waveguides may optically connect the FORJ to a centered waveguide about the rotational axis within the rotary probe. In some embodiments, a multi-use rotary waveguide may optically connect light from a centered waveguide about the rotational axis within the rotary probe directly to a rotary optical detector.

A rotatable probe may contain at least one (e.g., at least two) optical channel (e.g., a doubleclad fiber). In some embodiments, a rotatable probe may contain one illumination optical channel (e.g., a singlemode fiber) and one collection optical channel (e.g., a multimode fiber). In some embodiments, an illumination optical channel or a collection optical channel, or both, may have beam refracting and/or reflecting (e.g., mirrors) and/or shaping (e.g., lenses, e.g., non-flat reflective surfaces) components in their optical paths, between the transporting waveguides and a sample.

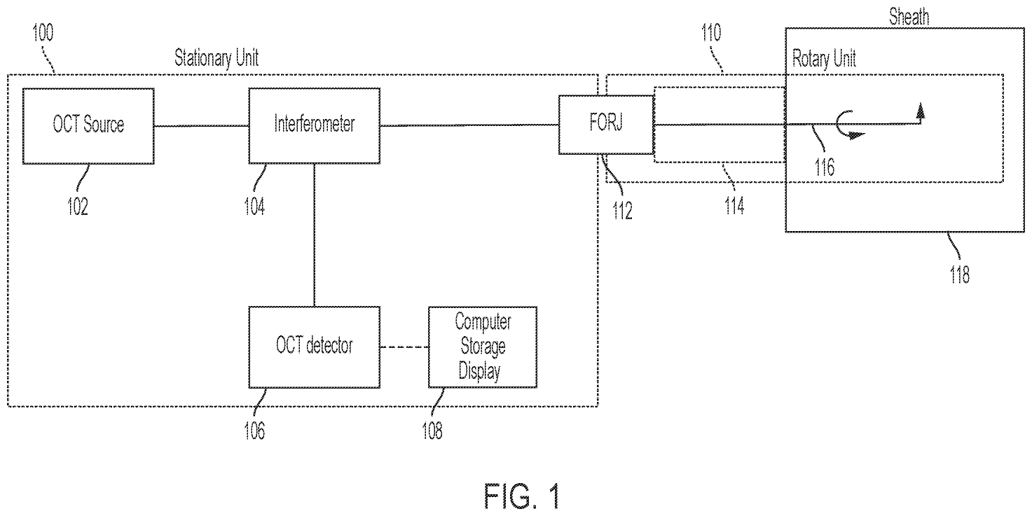

As a comparator, FIG. 1 shows a block diagram of a conventional OCT (e.g., optical frequency domain imaging (OFDI)) imaging system/apparatus. Such an imaging system consists of a stationary optical system 100, which may include an OCT source 102, an interferometer 104, an OCT detector arrangement 106 and a computer storage device 108. The stationary system 100 can be interfaced with a rotary optical system 110 via a FORJ 112. The rotary optical system 110, for example, can include a rotary device housing 114 and an optical channel 116 (e.g., at least one single mode fiber in series). Such a system transmits light from the OCT source 102 to a sample and receives back-reflected/scattered signal on the OCT detector 106 via the same optical channel, in order to acquire characterization results of the sample, and to process/store/display the results using a computer/storage device/assessment device 108. Optical transmission is indicated by solid lines, while electrical is depicted with dashed lines. Optical channels may comprise one or more waveguides coaxially (e.g., on the same optical path) disposed (e.g., a singlemode fiber, e.g., a double clad fiber).

In the figures, optical transmission (along optical path(s)) is indicated by solid lines while electrical transmission is indicated by dashed lines. Optical channels may comprise one or more waveguides coaxially disposed (e.g., a singlemode fiber, e.g., a double clad fiber), for example where each coaxial waveguide is used for as an illumination and/or collection channel for a different characterization modality or each coaxial waveguide is used as either an illumination channel or a collection channel for a single characterization modality. Signal (e.g., electrical) communications are indicated by dashed lines. Dotted boxes represent conceptual sections of a device and are provided for illustration only.

As shown in FIG. 1, a single channel FORJ may be used to transmit and receive light at high rotational speeds that require only a single optical channel, such as is found in single characterization modality systems. The present disclosure describes, among other things, how to achieve multimodal characterization, at high rotational speeds, with systems that require multiple channels (e.g., systems that perform more than one characterization modality).

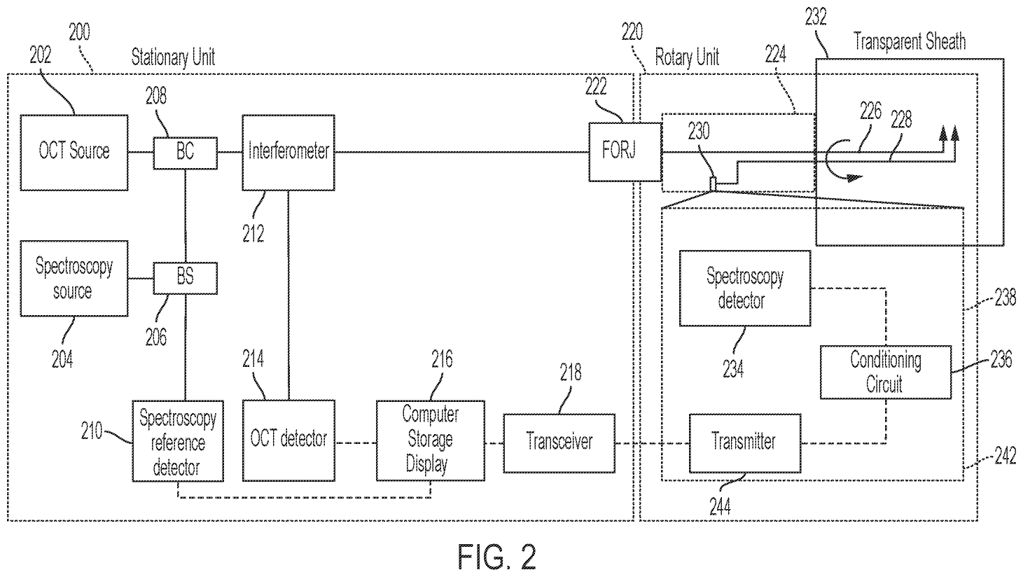

FIG. 2 is a block diagram illustrating exemplary embodiments of the present disclosure. The multimodal system/apparatus includes a stationary optical unit 200, which may include one or more of an OCT source 202, a spectroscopy source 204, a beam splitter 206 to create a reference channel, a beam combiner (e.g., a WDM) 208 to combine the two sources, a spectroscopy reference detector 210 for detecting a reference measurement, an interferometer 212, an OCT detector 214, a computing device 216 and a transceiver device 218. The stationary system 200 can be optically interfaced with a rotary optical unit 220 via a single channel FORJ 222. The rotary optical unit 220 may include, for example, one or more of a rotary device housing 224, an illumination optical channel (e.g., at least one singlemode fiber in series) 226, a collection optical channel (e.g., at least one multimode fiber in series) 228 and a collection light detector 230. The optical channels extending to the sample may be covered by a transparent sheath 232 for protection and safety considerations. The sheath 232 may remain stationary during operation, for example as in an intravascular characterization catheter. The collection detector device 230 may include a spectroscopy detector 234 as well as a conditioning circuit 236 (e.g., for improving the SNR of the detected signal), and a transmitter 244 (e.g., a slip ring, RF transmitter, or optical transmitter). Transmitter 244 may be a transceiver.

The system of FIG. 2 may transmit light from both the OCT source 202 and the spectroscopy source 204 to a sample and may receive sample-interacted light from both sources on both the OCT detector 214 (e.g., interferometric detection), through the illumination optical channel, as well as the spectroscopy detector 234 (e.g., reflectance detection) through the collection optical channel. The spectroscopy detector 234 may convert the signal to an electrical format and may further undergo signal preservation modifications within the rotary unit 220. The signal may then be transmitted to a receiver, for example in order to process/store/display the results using a computer/storage device/assessment device 218 on the stationary unit 200. In some embodiments, transmission occurs to a remote computing device (e.g., not part of stationary unit 200).

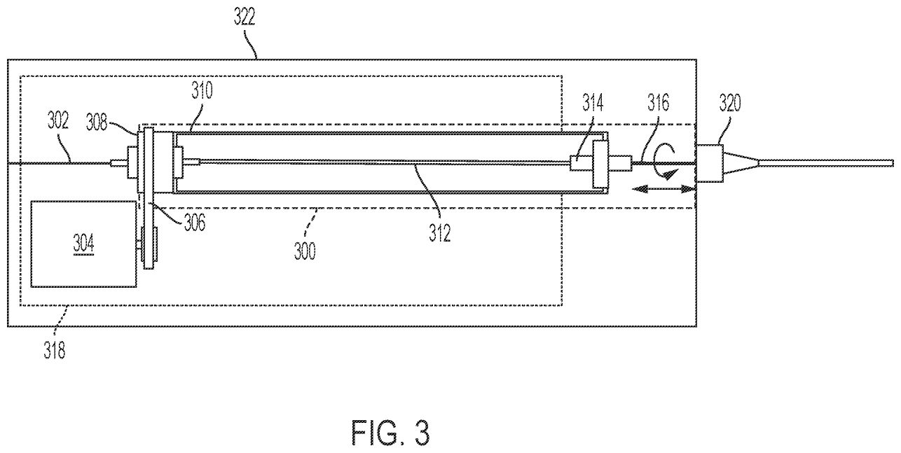

As a comparator, in FIG. 3, an example of a conventional rotary unit for single-modality characterization is shown. Such an exemplary rotary unit 300 consists of a drive belt 306, a FORJ 308, a rotary device housing 310 for housing a multi-use waveguide 312 and an interconnect 314, in order to connect to a single-use single-optical-channel probe 316. The entire rotary device may sit on a linear translational stage 318, allowing for a forward and backward translation. A probe housing 320 may be designed to connect to a stationary housing 320 for the rotary unit, therefore remaining stationary while the inner optical probe 316 is rotated and translated. Stationary connection fiber 302 connects FORJ 308 to a stationary unit. Drive belt 306 connects to motor 304 (that stays stationary in relation to a rotating probe 316).

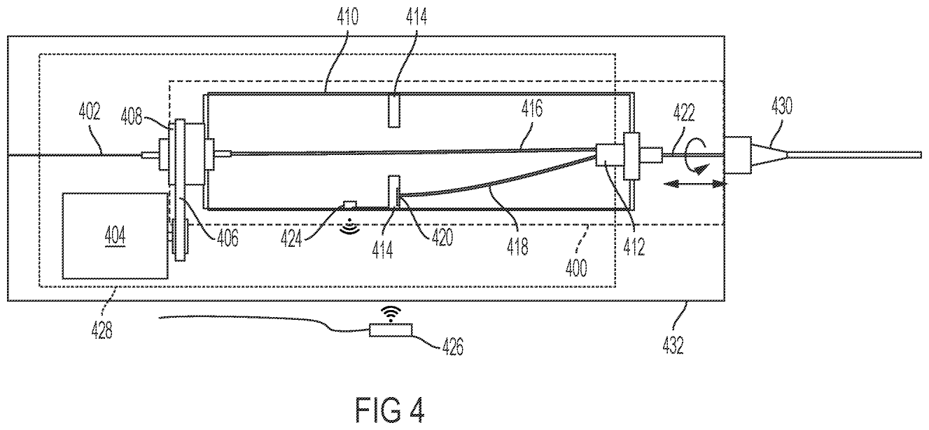

FIG. 4 is a cross section that illustrates exemplary embodiments for a rotary unit that enables high-fidelity, rapid multimodal rotary characterization. A rotary unit 400 may include a FORJ 408 (e.g., portion thereof), a rotary device housing 410 for housing an interconnect 412, a circuit board 414, an illumination optical channel 416, collection optical channel 418 terminating on an optical detection device 420 (e.g., a light detector, optionally including optics) and a multi-optical-channel probe 422 for transmitting and receiving light from a sample. A stationary unit may optically interface with a rotary unit 400 via a stationary optical fiber 402 interfaced with the FORJ 408. A motor 404 that stays stationary in relation to the rotary unit may be used to drive a drive belt 406 in order to transfer torque to the rotary unit 400. In some embodiments, a direct drive motor may be deployed without a drive belt. Within the rotary device housing 410, connected to the circuit board 414, there may exist a wireless transmitter 424 (e.g., a wireless transceiver) to transmit data and/or receive information from the rotary unit to a wireless RF transceiver 426 on the stationary unit. At least a portion of the rotary unit may be attached to a linear translational stage 428, allowing for a forward and backward translation. A probe housing 430 may be designed to house the optical probe 422 and to interface with a stationary housing 432 for the rotary devices, such as to remain rotationally stationary while the inner optical probe 422 is rotated and translated as part of the rotary unit 400. Housings and devices shown may be, for example, round (e.g., circular) or rectangular.

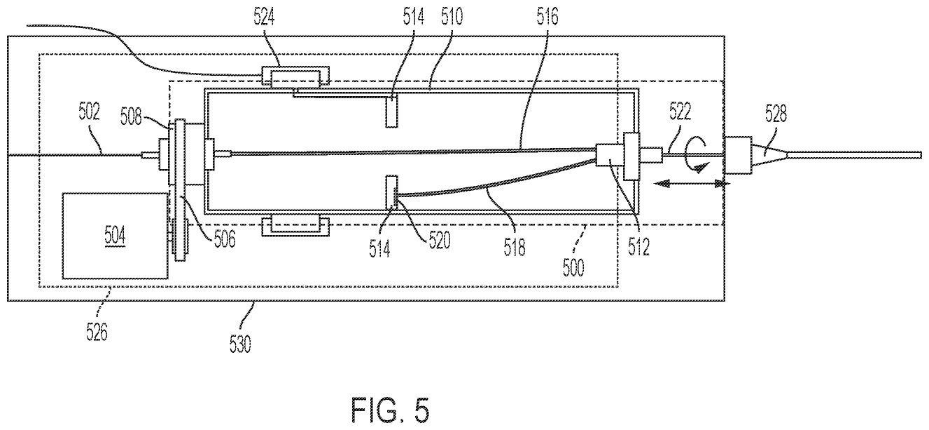

FIG. 5 is a cross section illustrating exemplary embodiments of a rotary unit that enables high-fidelity, rapid multimodal rotary characterization. A rotary unit 500 may include one or more of a FORJ 508 (e.g., portion thereof), a rotary device housing 510 for housing an interconnect 512, a circuit board 514, an illumination optical channel 516, collection optical channel 518 terminating on an optical light detector 520 and a multi-optical-channel probe 522 for transmitting and receiving light from a sample. A motor 504 that stays stationary in relation to the rotary unit may be used to drive a drive belt 506 in order to transfer torque to the rotary unit 500. In some embodiments, a direct drive motor may be deployed without a drive belt. A stationary unit may optically interface with a rotary unit 500 via a stationary optical fiber 502 interfaced with the FORJ 508. On the exterior of the rotary device housing 510 there may exist an electrical rotary joint 524 (e.g., an electrical slip ring) that transmits electrical signal between stationary and rotary contacts. Electrical rotary joint 524 may be disposed on an exterior surface of rotary device housing 510. At least a portion of the rotary unit may be attached to a linear translational stage 526, allowing for a forward and backward translation. A probe housing 528 may be designed to house the optical probe 522 and to interface with a stationary housing 530 for the rotary devices, such as to remain stationary while the inner optical probe 522 is rotated and translated as part of the rotary unit 500. Given the cross-sectional nature of the exemplary view, it is understood that housings and devices may be circular, rectangular, or any other advantageous shape. Illumination optical channel 516 may be used an optical collection channel for a second characterization modality, for example if a second detector is included in the stationary unit.

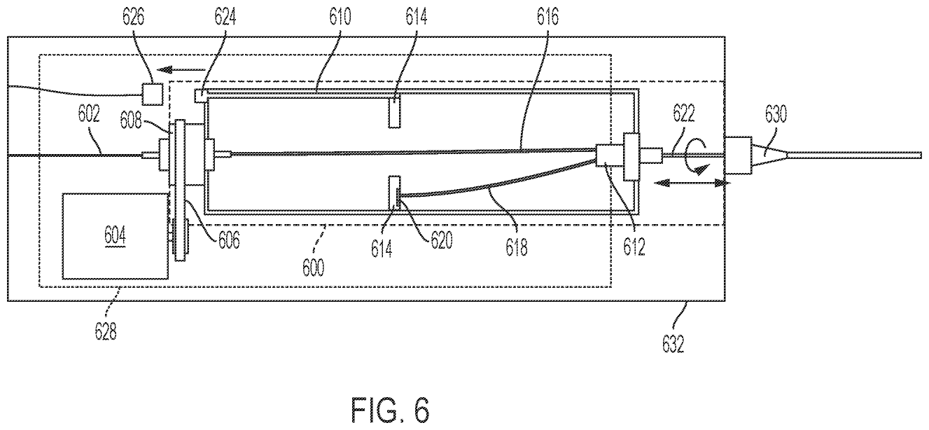

FIG. 6 is a cross section illustrating exemplary embodiments of a rotary unit that enables high-fidelity, rapid multimodal rotary characterization. A rotary unit 600 may include one or more of a FORJ 608 (e.g., portion thereof), a rotary device housing 610 for housing an interconnect 612, a circuit board 614, an illumination optical channel 616, collection optical channel 618 terminating on an optical detection device 620 (e.g., a detector with optics) and a multi-optical-channel probe 622 for transmitting and receiving light from a sample. A stationary unit may optically interface with a rotary unit 600 via a stationary optical fiber 602 interfaced with the FORJ 608. A motor 604 that stays stationary in relation to the rotary unit may be used to drive a drive belt 606 in order to transfer torque to the rotary unit 600. In some embodiments, a direct drive motor may be deployed without a drive belt. On the rotary device housing 610, connected to the circuit board 616, there may exist a storage component connected to an electrical contact 624 that can physically contact a stationary electrical contact 626 in order to transmit data and/or receive information to/from a rotary unit to/from a stationary unit (indicated by the black arrow) at specific translational positions while the rotary unit is stationary. At least a portion of the rotary unit may be attached to a linear translational stage 628, allowing for a forward and backward translation. A probe housing 630 (e.g., sheath) may be designed to house the optical probe 622 and to interface with a stationary housing 632 for the rotary devices, such as to remain rotationally stationary while the inner optical probe 622 is rotated and translated as part of the rotary unit 600.

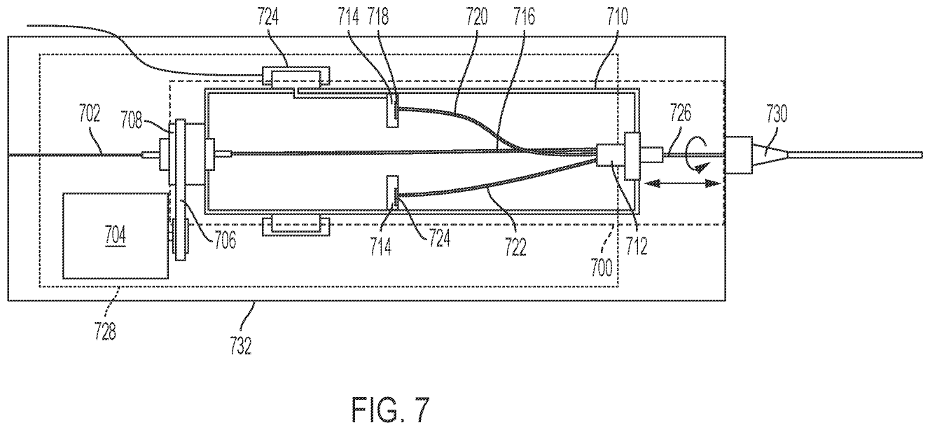

FIG. 7 is a cross section illustrating exemplary embodiments for a rotary unit that enables high-fidelity, rapid multimodal rotary characterization. A rotary unit 700 may include a drive belt 706, a FORJ 708 (e.g., portion thereof), a rotary device housing 710 for housing an interconnect 712, a circuit board 714, an illumination optical channel 716, a light source 718, a second illumination optical channel 720, a collection optical channel 722 terminating directly on an optical detection device 724 and a multi-optical-channel probe 726 for transmitting and receiving light from a sample. A stationary unit may optically interface with a rotary unit 700 via a stationary optical fiber 702 interfaced with the FORJ 708. A motor 704 that stays stationary in relation to the rotary unit may be used to drive a drive belt 706 in order to transfer torque to the rotary unit 700. In some embodiments, a direct drive motor may be deployed without a drive belt. On the rotary device housing, connected to the circuit board 614, there may exist an electrical rotary joint (e.g., rotary transformer, e.g., inductive coupling device) 724 that transmits electrical signal from (e.g., and to) the rotary unit, and optionally provides power to the rotary unit. At least a portion of the rotary unit may be attached to a longitudinally translational stage 730, allowing for a forward and backward translation. A probe housing 732 may be designed to house the optical probe 726 and to interface with a stationary housing 732 for the rotary devices, such as to remain rotationally stationary while the inner optical probe 726 is rotated and translated as part of the rotary unit 700.

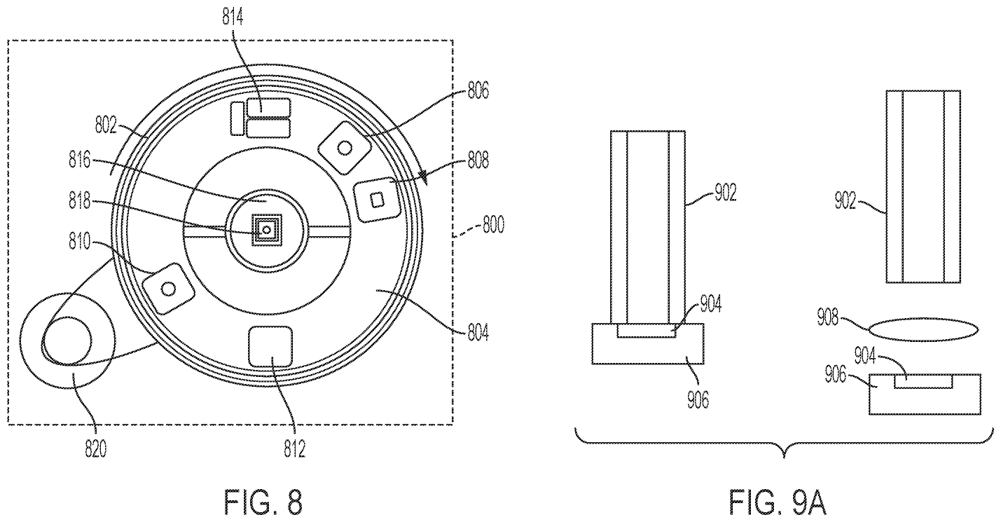

FIG. 8 is a cross section illustrating exemplary embodiments for a rotary unit that enables high-fidelity, rapid multimodal rotary characterization. A rotary unit 800 may include a rotary device housing 802 for housing an annular circuit board 804, a first detector 806, a second detector 808, an illumination source 810, a wireless transceiver 812 and a processing unit 814. Due to the annular structure of the circuit board, looking through the hollow center a FORJ 816 and an optical channel interconnect 818 can be seen. A motor 820 is connected to the rotary device housing to provide rotational torque. In some embodiments, only one detector, only an illumination source, or only one detector and an illumination source are disposed on a circuit board. The circuit board 804 is shown as annular (e.g., in order to provide weight balance) but is not necessarily annular. Moreover, a second circuit board may be included in some embodiments, for example another annular circuit board disposed in a different plane from the circuit board 804 or multiple circuit boards disposed in a common cross sectional plane (e.g., each forming an arc of an annulus).

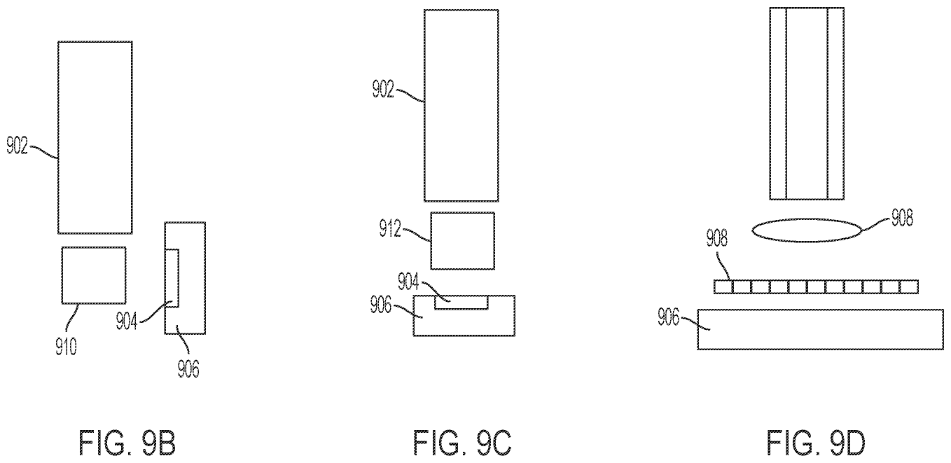

FIG. 9A illustrates exemplary embodiments for coupling optical channels to rotary optical detectors. On the left, an optical channel 902 is optically coupled with an active area 904 of an optical detector device 906, without any beam altering optics in the optical path between the optical channel and the detector. In the arrangement on the left, the optical channel is in physical contact with the detector, although it may also be simply placed near the optical detector. On the right, an optical channel 902 is optically coupled with an active area 904 of an optical detector device 906, using a lens 908 placed inbetween the optical channel 902 and the detector 906. In some embodiments, a fiber may optically couple a detector without being in-line with it (e.g., a right-angle mirror disposed between). The optics of FIG. 9B include beam splitter 910 (or mirror) to facilitate locating the detector 906 at a right angle relative to the optical channel 902. Other angles can be used depending on preferred arrangement of the components. The optics of FIG. 9C include dichroic/filter 912, which may be useful for, for example, fluorescence characterization modalities. The optics of FIG. 9D include a fiber Bragg grating 914 for spectral separation and a camera 916 as a detector, which may be useful for, for example, broadband spectroscopy or Raman spectroscopy characterization modalities.

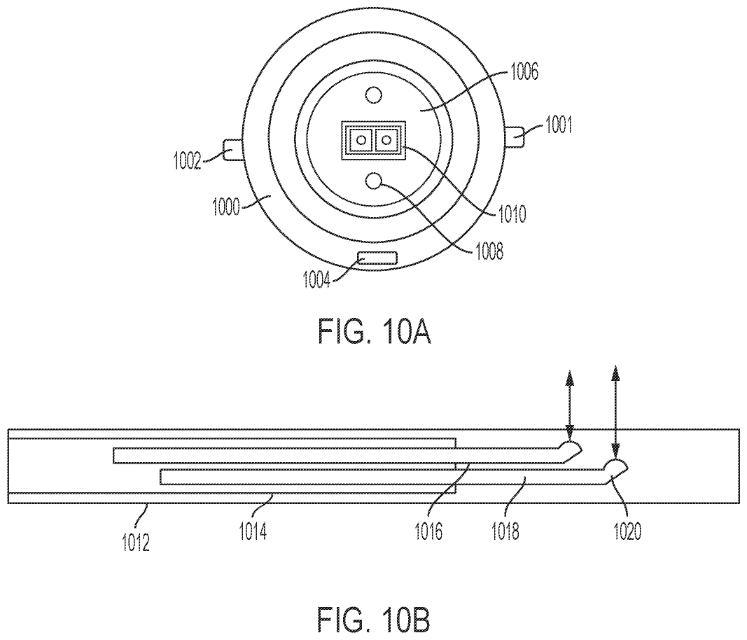

FIG. 10A illustrates a proximal end of a catheter used for characterization in accordance with exemplary embodiments of the present disclosure. On the proximal end of a catheter there is a stationary portion 1000 connectable, via an interlock mechanism 1002, to a stationary housing for the rotary unit. In the current illustration, an RFID 1004 for storing information is also on the stationary portion of the catheter. At the proximal end of the catheter there is also a rotatable portion 1006 connectable, via another interlock mechanism 1001, to a rotary unit. Also, on the rotatable portion of the proximal end of the catheter, a multi-channel interconnect 1010 is shown, allowing the extension of the optical channels from within the rotary unit to the distal end of the catheter. The rotary unit may provide rotational torque to the rotary portion of the catheter in order to rotate the optical channels and perform rotational characterization of a sample. In some embodiments, once connected, it is understood that the rotating portion of the catheter would be considered part of the rotary unit.

FIG. 10B illustrates a distal end of a catheter used for characterization in accordance with exemplary embodiments of the present disclosure. Depicted is a rotationally stationary sheath 1012, a rotatable drive shaft 1014, a first optical channel 1016 and a second optical channel 1018. At the tip of the optical channels, there may be beam re-directing and/or focusing optics to image circumferentially during rotation. Such optics are illustrated here in the form of angled ball lenses 1020 at the tip of each optical channel.

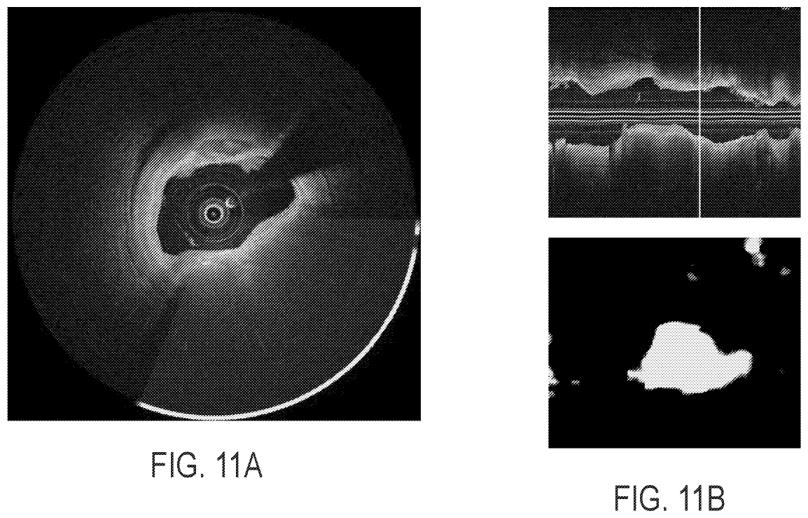

FIG. 11A shows an example of a multimodal image of a sample that was concurrently characterized with multiple modalities during a rotation of a rotary optical system used for characterization in accordance with exemplary embodiments of the present disclosure. The modalities used to form the image are OCT and NIRS spectroscopy. In the current example, OCT is used to provide a structural image of the sample based on its depth-dependent scattering properties while the NIRS is used to localize structures of interest (e.g., lipids) within the sample based on its bulk scattering and absorption properties. In the current example, NIRS lipid detection is both overlayed on the OCT image with an angular, lighthouse style, contrast as well as angularly indicated at the extremity of each OCT line where lipid is detected.

FIG. 11B shows an example of a 3 dimensional multimodal dataset of a sample that was concurrently characterized with multiple modalities during a rotation and a pullback of a rotary optical system used for characterization in accordance with exemplary embodiments of the present disclosure. The top image shows a single slice of the OCT image volume along the pullback, where the slice is located near the center of the imaging probe. The bottom image shows a flattened 2-dimensional representation of an output of an algorithm for analyzing NIRS data, where bright denotes higher lipid signal and dark denotes lower lipid signal. The OCT data acquires 3-dimensional data wherein each dimension comprises a structural/locational dimension. The NIRS data is also 3-dimensional but only two of the dimensions comprise structural/locational information while the other dimension comprises a NIRS spectra, or rather a spectral dimension. In the current example, the NIRS data shows the results of an algorithm designed to convert the spectral dimension into an indicator of lipid, based on the spectral dimension information.

The multimodal data shown in FIG. 11A-B were taken at a rotational speed greater than 10,000 rpm and a pullback speed of greater than 30 mm/s, consistent with state-of-the-art single modality OCT systems, which was achievable using a rotary unit in accordance with embodiments of the present disclosure. As shown, the OCT modality preserves its theoretical high sensitivity (>100 dB) as well as high axial (10 μm) and lateral resolution (40 μm), unaffected by the addition of the NIRS modality. The ability to perform rapid multimodal characterization without sacrificing performance of the OCT modality is a key strength of embodiments of the disclosed technology.

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems according to certain embodiments of the present disclosure that consist essentially of, or consist of, the recited components, and that there are processes and methods according to certain embodiments of the present disclosure that consist essentially of, or consist of, the recited processing steps.

It should be understood that the order of steps or order for performing certain action is immaterial so long as operability is not lost. Moreover, two or more steps or actions may be conducted simultaneously.

It should be understood that the exemplary procedures described herein can be stored on any computer accessible medium, including a hard drive, RAM, ROM (e.g., EPROM or EEPROM), removable disks, CD-ROM, memory sticks, etc., and executed by a processing arrangement and/or computing arrangement which can be and/or include a processor, microprocessor, mini, macro, mainframe, etc., including a plurality and/or combination thereof.

The foregoing merely illustrates the principles of the disclosure. Various modifications and alterations to the described embodiments will be apparent to those skilled in the art in view of the teachings herein.

The arrangements, systems and methods according to the exemplary embodiments of the present disclosure can be used with and/or implement any and/or be implemented into any OCT system, OFDI system, SD-OCT system, TD-OCT system, or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties.

What is claimed is:

1. A characterization system comprising:
a stationary unit optically connected to a rotary unit,
wherein the rotary unit comprises a first optical channel, a second optical channel and a first detector (e.g., first light detector),
wherein the stationary unit comprises a second detector (e.g., second light detector), and
wherein the first optical channel is optically connected to the first detector for detecting light for a first characterization modality and the second optical channel is optically connected to the second detector for detecting light for a second characterization modality.

2. The system of claim 1, comprising two light sources (e.g., at least three light sources).

3. The system of claim 2, wherein the two light sources are optically connected to the second optical channel to provide illumination from the two light sources through the second optical channel (e.g., through a same waveguide or through different coaxial waveguides).

4. The system of claim 2, wherein only one of the two light sources is optically connected to the second optical channel to provide illumination from the only one of the two light sources.

5. The system of claim 4, wherein the rotary unit comprises a third optical channel and only one of the two light sources is optically connected to the third optical channel.

6. The system of any one of claims 2-5, wherein at least one of the two light sources is a swept source.

7. The system of any one of claims 2-6, wherein at least one of the two light sources is a broadband source.

8. The system of any one of claims 2-7, wherein at least one of the two light sources is a narrowband source.

9. The system of any one of claims 2-8, comprising a probe optically connected to the rotary unit (e.g., and wherein one of the two light sources is disposed at a tip of the probe).

10. The system of any one of claims 1-9, wherein the first optical channel is also optically connected to the second detector.

11. The system of any one of claims 1-10, wherein the first optical channel comprises a single mode waveguide and the second optical channel comprises a multimode waveguide or the first optical channel comprises a multimode waveguide and the second optical channel comprises a single mode waveguide.

12. The system of any one of claims 1-11, wherein at least one of the first optical channel and the second optical channel comprises more than one optical waveguide connected in series within the rotary unit.

13. The system of any one of claims 1-12, wherein the rotary unit comprises a wireless transmitter (e.g., transceiver) operable to wirelessly transmit data (e.g., analog or digital data) corresponding to signal detected by the first detector (e.g., wherein the wireless transmitter is an RF transmitter, an electrical slip ring, or an optical transmitter).

14. The system of any one of claims 1-13, wherein the rotary unit comprises an electrical transmitter (e.g., transceiver) operable to electrically transmit data corresponding to signal detected by the first detector (e.g., by brushes, or by docking the electrical transmitter).

15. The system of any one of claims 1-14, wherein the rotary unit is operable to transmit data corresponding to signal detected by the first detector during rotation of the rotary unit.

16. The system of any one of claims 1-15, wherein the rotary unit is operable to transmit data corresponding to signal detected by the first detector while (e.g., exclusively while) the rotary unit is stationary.

17. The system of any one of claims 1-16, wherein the rotary unit further comprises an information storage device operable to store data corresponding to signal detected by the first detector.

18. The system of any one of claims 1-17, wherein the rotary unit further comprises an energy storage device (e.g., a battery) to provide power.

19. The system of any one of claims 1-18, wherein the rotary unit comprises a circuit (e.g., a conditioning circuit, transimpedance amplifier, analog to digital converter, single to differential circuit, or combination thereof) operable to process (e.g., enhance) (e.g., in real time) signal detected by the first detector prior to transmission.

20. The system of claim 19, wherein the circuit is partially encapsulated by a noise-reducing electrical isolation device (e.g., a faraday cage, e.g., a metal casing, e.g., an aluminum block).

21. The system of any one of claims 1-20, wherein the rotary unit comprises a rotary detector housing in which the first detector is disposed and wherein the rotary detector housing is cylindrical.

22. The system of any one of claims 1-21, wherein the rotary unit comprises a circuit board and the first detector is disposed on the circuit board (e.g., further comprises another circuit board).

23. The system of claim 22, wherein (i) the circuit board and the first detector are together rotationally weight balanced around an axis, (ii) the rotary unit is rotationally weight balanced around an axis, or (iii) both (i) and (ii).

24. The system of claim 22 or claim 23, wherein the circuit board is circular (e.g., and is disposed around the second optical channel).

25. The system of any one of claims 1-24, wherein (i) the first characterization modality is optical coherence tomography, reflectance imaging, visible spectroscopy, NIRS, or Raman spectroscopy; (ii) the second optical channel is an illumination channel for the second characterization modality, which is different from the first characterization modality, (e.g., and a collection channel for the second characterization modality) and the second characterization modality is optical coherence tomography, reflectance imaging, visible spectroscopy, NIRS, or Raman spectroscopy; or (iii) both (i) and (ii).

26. The system of any one of claims 1-25, wherein the first optical channel is comprised in a first characterization modality subsystem for the first characterization modality and the second optical channel is comprised in a second characterization modality subsystem for the second characterization modality, which is different than the first characterization modality.

27. The system of claim 26, wherein at least one of the first modality subsystem and the second modality subsystem comprises a single photodetector (e.g., wherein the first detector comprises a single photodetector).

28. The system of claim 26 or claim 27, wherein at least one of the first modality subsystem and the second modality subsystem comprises multiple photodetectors (e.g., wherein the first detector comprises multiple photodetectors).

29. The system of any one of claims 26-28, wherein at least one of the first modality subsystem and the second modality subsystem comprises a camera (e.g., a CMOS camera) (e.g., wherein the camera comprises the first detector).

30. The system of any one of claims 26-29, wherein at least one of the first modality subsystem and the second modality subsystem comprises an interferometer (e.g., wherein the interferometer comprises the first detector).

31. The system of claim 26, wherein at least one of the first modality subsystem and the second modality subsystem comprises a spectral separation device disposed prior to a detector (e.g., wherein a spectrometer comprises the first detector).

32. The system of claim 26, wherein the second optical channel is an illumination channel for the first characterization modality and the second characterization modality.

33. The system of claim 32, wherein the second optical channel is a collection channel for the second characterization modality.

34. The system of claim 1, wherein the first optical channel is a collection channel for the first characterization modality and the second optical channel is an illumination channel for at least the first characterization modality.

35. The system of any one of claims 1-34, wherein rotary unit comprises a light source and a third optical channel that is optically connected to the light source.

36. The system of any one of claims 1-35, wherein at least a portion of each of the first channel and the second channel are sized and shaped to characterize intravascular lumens.

37. The system of any one of claims 1-36, wherein the rotary unit is operable to obtain measurements for the first characterization modality at a spatial sampling rate greater than 10 kHz.

38. The system of any one of claims 1-37, comprising a sheath, wherein at least a portion of the rotary unit (e.g., comprising a probe, e.g. optically connected to the first optical channel and the second optical channel by an interconnect) is disposed in the sheath (e.g., wherein the sheath remains stationary during rotation of the rotary unit).

39. The system of any one of claims 1-38, wherein the rotary unit comprises an interconnect and the first optical channel and the second optical channel are optically connected to the interconnect.

40. The system of claim 39 wherein the rotary unit has a distal end that is closest to where sample characterization occurs and the interconnect is disposed at the distal end.

41. The system of claim 39 or claim 40, wherein the rotary unit comprises at least a portion of a FORJ and at least a portion of the second optical channel is disposed between the interconnect and the FORJ.

42. The system of claim 41, wherein the FORJ and the interconnect are disposed at opposing ends of the rotary unit (e.g., opposing ends of a rotary unit housing).

43. The system of claim 42 or claim 43, wherein the first detector is disposed in the rotary unit between the FORJ and the interconnect.

44. The system of any one of claims 41-43, wherein the second optical channel is physically connected at one end to the FORJ and at an opposing end to the interconnect.

45. The system of any one of claims 41-44, wherein the first optical channel is physically connected at one end to the interconnect and at an opposing end to the first detector.

46. The system of any one of claims 1-45, wherein the characterization system comprises a catheter (e.g., a cardiac catheter).

47. The system of any one of claims 1-46, further comprising a light source that is constructed and arranged to emit light in a wavelength band comprising (e.g., centered around) a characterization peak for characterizing arterial plaque.

48. The system of any one of claims 1-46, wherein the second characterization modality is different from the first characterization modality and the second characterization modality is optical coherence tomography.

49. The system of claim 48, wherein the first characterization modality is reflectance imaging, fluorescence spectroscopy, visible spectroscopy, NIRS, or Raman spectroscopy.

50. The system of any one of claims 1-49, wherein the rotary unit rotates at greater than 3,500 rpm, greater than 5,000 rpm, or greater than 6,000 rpm during operation.

51. The system of any one of claims 1-50, wherein the rotary unit rotates at greater than 10,000 rpm during operation.

52. The system of any one of claims 1-51, wherein the system has a characterization sensitivity of greater than 100 dB for at least one of the first characterization modality and the second characterization modality.

53. The system of any one of claims 1-51, wherein the system has a characterization sensitivity of greater than 90 dB, greater than 95 dB, or greater than 110 dB for at least one of the first characterization modality and the second characterization modality.

54. A method for rotational sample characterization, the method comprising:
providing illumination light to a sample through a second optical channel;
rotating (e.g., by a motor) the second optical channel, a first optical channel, and a first light detector; and
collecting, during the rotating, first signal from the sample through the first optical channel with the first light detector.

55. The method of claim 54, comprising collecting (e.g., during the rotating) second signal from the sample through the second optical channel with a second (e.g., stationary) light detector.

56. The method of claim 55, comprising characterizing the sample with a first modality (e.g., NIRS) using the signal collected by the first light detector and characterizing the sample with a second modality (e.g., OCT) using the second signal collected by the second light detector (e.g., a characterization sensitivity for at least one of the first characterization modality and the second characterization modality is greater than 100 dB) (e.g., a characterization sensitivity for at least one of the first characterization modality and the second characterization modality is greater than 90 dB, greater than 95 dB, or greater than 110 dB).

57. The method of any one of claims 54-56, comprising characterizing the sample with a first modality using at least a portion of the signal collected with the first light detector and at least a portion of the signal collected with the second light detector.

58. The method of any one of claims 54-57, wherein rotating the first optical channel and the first light detector comprises rotating the first light detector about the second optical channel.

59. The method of any one of claims 54-58, wherein rotating the first optical channel, the second optical channel, and the first light detector further comprises rotating a light source and a third optical channel and the method comprises providing illumination light from the light source through the third optical channel during the rotating.

60. The method of any one of claims 54-59, comprising (e.g., wirelessly) transmitting data corresponding to the signal while the rotating is occurring.

61. The method of any one of claims 54-59, comprising transmitting data corresponding to the signal after ending the rotating.

62. The method of any one of claims 54-61, wherein collecting the signal comprises splitting light received from the sample such that a portion of the light travels through the first optical channel.

63. The method of any one of claims 54-62, comprising obtaining measurements from the signal at a spatial sampling rate greater than 10 kHz.

64. The method of any one of claims 54-63, wherein the rotating occurs at a rate of greater than 3,500 rpm, greater than 5,000 rpm, or greater than 6,000 pm.

65. The method of any one of claims 54-64, wherein the rotating occurs at a rate of greater than 10,000 rpm.

66. A characterization system, comprising:
optionally, a stationary unit; and
a rotary unit, optionally optically connected to the stationary unit (e.g., via a FORJ), the rotary unit comprising a first optical channel, a second optical channel (e.g., wherein the stationary unit is optically connected to the rotary unit at least in part with the second optical channel) (e.g., comprising a multimode fiber and/or multiple waveguides), and a light source for providing illumination light, wherein the first optical channel is optically connected to the light source.

67. The system of claim 66, wherein the rotary unit further comprises a light detector.

68. The system of claim 66 or claim 67, further comprising a means to wirelessly transmit a detected signal from the rotary portion to the stationary portion.

69. The system of any one of claims 66-68, further comprising a means to transmit a detected signal from the rotary portion to the stationary portion during rotation of the rotary portion by physical contact.

70. The system of any one of claims 66-69, wherein the rotary unit further comprises an energy storage device (e.g., a battery).

71. A rotary unit for a sample characterization system, the unit comprising:
a rotatable housing;
a circuit board and a light detector or light source disposed on the circuit board, wherein the circuit board is attached to the housing (e.g., an interior or exterior of the housing);
a first optical channel optically connected to the light detector or light source; and
a second optical channel disposed through the housing along an axis, wherein the circuit board is at least partially around the axis.

72. The unit of claim 71, wherein the printed circuit board and the light detector or light source are weight balanced with respect to the axis.

73. The unit of claim 72 or claim 72, wherein the circuit board is partially encapsulated by a noise-reducing electrical isolation device (e.g., a faraday cage, e.g., an aluminum block).

74. The unit of any one of claims 71-73, wherein the circuit board and the housing each have a circular cross section.

75. A multimodality optical device having a proximal face and a distal face, a proximal optical port on the proximal face, and a distal optical port on the distal face, comprising:
a first optical waveguide configured to receive and transmit a first characterization modality, the first optical waveguide making an optical connection between the proximal optical port and the distal optical port; and
a second optical waveguide, configured to receive and transmit a second characterization modality, the second optical waveguide optically connected to the distal optical port, and optically connected to a detector (e.g., housed within a rotary unit of the device).

76. A characterization system comprising:
optionally, a stationary unit; and
a rotary unit, optionally optically connected to the stationary unit (e.g., via a FORJ), the rotary unit comprising a first optical channel, a second optical channel (e.g., wherein the stationary unit is optically connected to the rotary unit at least in part with the second optical channel) (e.g., comprising a singlemode fiber and/or multiple waveguides), and a light detector for detecting light for a first characterization modality (e.g., a camera, interferometer, or spectrometer),
wherein the first optical channel is optically connected to the light detector (e.g., and wherein the second optical channel is used to detect light for a second characterization modality).
